(19) United States
(12) Patent Application Publication
(10) Pub. No.: US 2012/0156199 A1
(43) Pub. Date: Jun. 21, 2012

(54) USE OF PICOPLATIN TO TREAT COLORECTAL CANCER

(75) Inventors: David A. Karlin, Los Altos, CA (US); Hazel B. Breitz, Seattle, WA (US); Paul L. Weiden, Seattle, WA (US)

(73) Assignee: Poniard Pharmaceuticals, Inc., Seattle, WA (US)

(21) Appl. No.: 13/319,852

(22) PCT Filed: May 12, 2010
(86) PCT No.: PCT/US10/34593

§ 371 Date: Mar. 1, 2012

Related U.S. Application Data
(63) Continuation-in-part of application No. 12/367,394, filed on Feb. 6, 2009.
Application No. 12/367,394 is a continuation-in-part of application No. 11/982,841, filed on Nov. 5, 2007, now abandoned.
(60) Provisional application No. 60/857,066, filed on Nov. 6, 2006.
(60) Provisional application No. 60/857,725, filed on Nov. 8, 2006.
(60) Provisional application No. 60/877,495, filed on Dec. 28, 2006.
(60) Provisional application No. 60/889,191, filed on Feb. 9, 2007.
(60) Provisional application No. 60/931,589, filed on May 24, 2007.
(60) Provisional application No. 60/983,852, filed on Oct. 30, 2007.

(51) Int. Cl. A61K 39/395; A61P 35/04; A61P 35/00

(52) U.S. Cl. 424/133.1

(57) ABSTRACT

The invention provides a method of treatment of colorectal cancer by administration of the anti-cancer platinum drug picoplatin in conjunction with 5-FU and leucovorin in a variety of treatment regimens.

### CROSS REFERENCES TO RELATED APPLICATIONS

This application is claims priority to U.S. application Ser. Nos. 12/464,662, filed May 12, 2009, and 12/465,563, filed May 13, 2009. This application is also a continuation-in-part of Ser. No. 12/367,394 filed Feb. 6, 2009, which is in turn a continuation-in-part application of U.S. application Ser. No. 11/982,841, filed Nov. 5, 2007, which claims the priority of U.S. Provisional Patent Applications, Ser. Nos. 60/857,066 (filed Nov. 6, 2006), 60/857,725 (filed Nov. 8, 2006), 60/877,495 (filed Dec. 28, 2006), 60/889,191 (filed Feb. 9, 2007), 60/931,589 (filed May 24, 2007), and 60/983,852 (filed Oct. 30, 2007), all of which are incorporated by reference herein in their entireties.

### BACKGROUND OF THE INVENTION

Colorectal cancer remains the second most common cause of cancer-related death in the United States and a significant cause of cancer-related death in other countries as well.¹ For decades, the only approved chemotherapeutic drug for treatment of colorectal cancer was 5-fluorouracil (5-FU), and it continues to be the backbone of most first-line chemotherapeutic regimens for patients with advanced disease. However, there has been much progress made in treatment of colorectal cancer in the past decade, with the approval of several new therapeutic agents including irinotecan, oxaliplatin, capecitabine, and most recently, cetuximab and bevacizumab.^{2,3} Importantly, a variety of new chemotherapeutic regimens utilizing these agents have been devised, which have led to increased response rates and incremental increases in the time to progression and median survival for patients with advanced disease.^{2,3} Response rates for 5-FU/leucovorin, irinotecan, and oxaliplatin as single agent therapy have been low (23%, 18%, and 12%, respectively), progression-free survival has been short (median 4.0, 4.3, and 4.0 months, respectively), and median survival has also been short, approximately (12 , 12, and 14.5 months, respectively).⁴ With the introduction of 5-FU-based combination chemotherapeutic regimens using irinotecan and oxaliplatin, the response rate has increased substantially, with response rates reported as high as 64% (FOLFOX7), time to progression ranging from 8.9-12.3 months, and median survival now approaching approximately 20 months in some reports.²⁻⁴

Unfortunately, however, these newer combination chemotherapy regimens do have increased toxicity. Regimens containing irinotecan are associated with significant diarrhea and other gastrointestinal toxicity, while those containing oxaliplatin are associated with neurotoxicity.²⁻¹⁰ The neurotoxicity observed is of two types: first, a cumulative and often dose limiting sensory loss with paresthesias that can interfere with function and second, a disturbing cold sensitivity that limits patient acceptance of the FOLFOX regimen.⁷⁻¹⁰ Thus a drug of comparable efficacy without neurotoxicity would be a welcome substitute for oxaliplatin in combination with 5-FU and leucovorin.

Picoplatin is a platinum analogue that has demonstrated synergy with 5-FU in vitro in pre-clinical studies and has undergone extensive Phase 1 and 2 testing in a variety of cancers.¹¹⁻²² Like other platinum analogues, picoplatin causes cell death by the formation of covalent cross-links in DNA that interfere with DNA replication and transcription, leading to cell death. Cisplatin, the first platinum analogue, was introduced approximately 20 years ago and is still widely used. The approval of cisplatin was followed by approval of carboplatin, and most recently by that of oxaliplatin.

Treatment with platinum analogues is limited by their toxicity. While neurotoxicity and nephrotoxicity are the main dose-limiting toxicities (DLT) observed following cisplatin treatment, myelosuppression is most significant following carboplatin treatment. Carboplatin is known to cause cumulative dose-related toxicity that results in slow bone marrow recovery. Peripheral neurotoxicity is well documented in patients treated with oxaliplatin. The unacceptable nephrotoxicity, oto-, and neurotoxicity associated with earlier platinum analogues has not been reported with picoplatin either in animal studies or in clinical trials.^{11, 19-22}

The efficacy of platinum analogues is also limited by several (intrinsic or acquired) mechanisms of resistance, including impaired cellular uptake, intracellular inactivation by thiols [e.g., reduced glutathione], and enhanced DNA repair and/or increased tolerance to platinum-DNA adducts.²³ Pre-clinical studies indicate that picoplatin can overcome these three mechanisms of resistance. This has been demonstrated in vitro and by using human ovarian xenograft tumor models that exhibit resistance to cisplatin.¹³⁻¹⁷ Several human ovarian and colon cell lines with induced resistance to oxaliplatin retain sensitivity to picoplatin.¹⁶⁻¹⁸

In Phase 1 studies, indications of activity were seen in subjects with ovarian cancer, non-small cell lung cancer (NSCLC), small cell lung cancer (SCLC), colorectal cancer, head and neck cancer, renal cell cancer, thymic cancer, pancreatic cancer, stomach cancer, leiomyosarcoma, liver cancer, mesothelioma, and prostate cancers.^{24,25} In Phase 2 studies, indications of efficacy were seen in subjects with ovarian, NSCLC, SCLC, mesothelioma, prostate cancer, and breast cancer.

Picoplatin and processes for making picoplatin and for using picoplatin in treatment are disclosed and claimed in U.S. Pat. No. 5,665,771 (issued Sept. 9, 1997), and U.S. Pat. No. 6,518,428 (issued Feb. 11, 2003), and in PCT/GB0102060, filed May 10, 2001, published as WO2001/087313, which are incorporated herein by reference in their entireties.

About 40% of patients with mCRC have K-ras mutations and their mCRC does not respond to epidermal growth factor receptor (EGFR) inhibitors such as cetuximab and panitumumab. Many cetuximab-treatment studies in mCRC demonstrated very low or even zero response rates, short progression-free survival, and short overall survival in K-ras mutation positive mCRC. Because K-ras wild type CRC patients treated with EGFR inhibitors have significantly higher objective response rates, increased progression-free survival, and increased overall survival, K-ras testing is now used in routine clinical practice to select the subset of mCRC patients most likely to benefit from treatment with an EGFR inhibitor. Subset selection spares patients who are unlikely to respond to EGFR inhibitors for side effects and the cost of an ineffective drug. Examples of companies that offer K-ras testing to medical oncologists include:

For example, see: M. Brink et al., Carcinogenesis. 2003;24:703-10; A. Lièvre et al., J Clin Oncol. 2008;26:374-9; W. De Roock et al., Ann Oncol. 2007, Nov. 12; F. Di Fiore et al., Br J Cancer. 2007;96:1166-9; A. Lièvre et al., Cancer Res. 2006;66:3992-5; C. S. Karapetis et al., NEJM. 2008;359 (N 17):1757-1765; Amado et al., 2008 American Society of Clinical Oncology Gastrointestinal Cancers Symposium, Abstract 278.

Picoplatin (SP-4-3) (cis-aminedichloro(2-methylpyridine)Pt(II)), and useful prodrugs and analogs thereof are disclosed in U.S. Pat. Nos. 5,665,771; 6,518,428; 6,413,953 and PCT/GB/01/02060.

### SUMMARY OF THE INVENTION

An embodiment of the present invention provides a method of treatment of colorectal cancer, comprising administering to a patient afflicted with colorectal cancer picoplatin, 5-fluorouracil (5-FU), and leucovorin, wherein 5-FU and leucovorin are administered intravenously at least twice at intervals of about 2-6 weeks and the picoplatin is administered with the leucovorin and 5-FU every other time that the fluorouracil and leucovorin are administered.

Another embodiment of the invention provides a method of treatment of colorectal cancer, comprising administering to a patient afflicted with colorectal cancer effective amounts of a combination of picoplatin, 5-FU and leucovorin, wherein the picoplatin, 5-FU and leucovorin are administered intravenously at least twice at intervals of about two weeks, wherein the amount of picoplatin is less than the maximum tolerated dose of picoplatin when administered in said combination.

Another embodiment of the invention provides a method of treatment of colorectal cancer, comprising administering to a patient afflicted with colorectal cancer picoplatin, 5-FU, and leucovorin, wherein 5-FU and leucovorin are administered intravenously at intervals of about two weeks, and the picoplatin is administered with the leucovorin and 5-FU every time that the fluorouracil and leucovorin are administered, wherein the picoplatin is administered at a dose of about 45-120 mg/m².

In various embodiments of the present method, the patient has not previously been treated for metastatic disease, or the patient has not previously had systemic treatment, such as chemotherapy, for localized or metastatic disease. For example, the patient may have had surgery to remove, or to de-bulk the primary tumor or to resect the portion of the colon or rectum comprising the tumor and then can be treated with one of the picoplatin, 5-FU, leucovorin regimens (e.g., FOLPI) of the invention to treat any remaining primary tumors or metastatis, and/or to prevent or delay progression of the cancer, including to prevent or delay the development of metastases. The patient may have received earlier chemotherapy at the time of primary tumor treatment, e.g., at least 6 months prior to the present picoplatin treatment. Following picoplatin treatment, a patient, such as one responsive to picoplatin, may undergo resection of the primary lesion or metastases on other organs or tissues. In one embodiment, the primary tumor is resected or debulked following administration of picoplatin, 5-FU and leucovorin, while in another embodiment, the patient has previously (e.g., prior to administration of picoplatin, 5-FU and leucovorin) had surgery to resect or de-bulk a colorectal tumor.

In various embodiments, the picoplatin can be administered with curative intent, rather than merely seeking to arrest the disease with no remission. The dosage of the picoplatin can be increased beyond that bringing about disease stasis in order to achieve a cure in the patient, including complete response of both the primary colorectal tumor(s) and any metastases.

The picoplatin and the leucovorin can be administered concurrently in any treatment cycle where picoplatin is administered.

In another embodiment of the invention, the picoplatin is administered substantially concurrently with the leucovorin and the picoplatin is administered at every second treatment of the patient with the 5-FU and the leucovorin, e.g., every four weeks. The leucovorin can be administered at a dosage of about 250-500 mg/m², preferably at about 400 mg/m². The picoplatin is administered at a dosage of about 60-180 mg/m². The 5-FU is administered at a total dosage of about 2500-3000 mg/m². A preferred treatment cycle for leucovorin and 5-FU is every two weeks, and picoplatin is administered every 4 weeks, e.g., at a low dose of about 60-75 mg/m², e.g., 60 mg/m², or at a high dose of about 120-180 mg /m², preferably about 120-150 mg/m², e.g. about 150 mg/m².

Therefore, in one embodiment of the invention, the leucovorin, at a dosage of 250-500 mg/m², is administered as an about 2 hour infusion concurrently with the picoplatin, when it is given, wherein the picoplatin dosage is 120-180 mg/m², e.g., about 150 mg/m²; the administration of the leucovorin and the picoplatin being followed by a 5-FU dosage of about 400 mg/m² as a bolus; the 5-FU dosage being followed by 5-FU at a dosage of 2,400 mg/m², preferably administered as a 46 hour continuous infusion, wherein the leucovorin and 5-FU are provided to the patient at intervals of two weeks and the leucovorin, picoplatin, and 5-FU are provided to the patient at alternating intervals of four weeks. In another embodiment, a low dose of picoplatin of about 45-75 mg/m², e.g., about 60-75 mg/m², e.g., about 60 mg/m², is administered.

In another embodiment of the invention, the leucovorin, at a dosage of 400 mg/m², is administered as a 2 hour infusion; the administration of the leucovorin being followed by a 5-FU bolus at a dosage of 400 mg/m²; the 5-FU bolus dosage being followed by parenteral 5-FU at a dosage of 2,400 mg/m², preferably administered as a 46 hour continuous infusion; the administration of the leucovorin and the 5-FU taking place every two weeks; wherein every two weeks picoplatin, at a dosage of up to about 50 mg/m², e.g., at about 40-50 mg/m², e.g., about 45 mg/m², is administered concurrently with the leucovorin, preferably simultaneously. Picoplatin dosages of about 45-105 mg/m² can also be administered.

It has unexpectedly been found that, in some cases, the combination of low doses of picoplatin administered with leucovorin and 5-FU at every treatment cycle, are as effective as, or more effective than, higher doses, e.g., the MTD, given at the same intervals, in producing a response. The MTD for the 2 week and 4 week picoplatin administration schedules (see Table 1) are discussed below. Preferably, such doses in the initial treatment are lower or substantially lower than the MTD. Such doses can range from about 40-60 mg/m² of picoplatin every two weeks, given with leucovorin and followed by 5-FU, as discussed below.

It has surprisingly been found that a total cumulative dose in excess of about 900 mg/m² can be tolerated by patients without neuropathy of Grade 2 or higher being observed.

As used herein, the term “concurrently” means that the administrations are simultaneous, overlapping or close enough in time so that the two or more agents administered are present in vivo in therapeutically effective amounts.

The present method also can comprise administration of an effective amount of a 5-HT₃ receptor antagonist, as an anti-emetic.

The present invention also provides a method comprising administering picoplatin in a dosage form comprising an isotonic solution comprising water, a tonicity adjuster, and about 0.5 mg/mL dissolved picoplatin. The dosage form can also comprise an effective amount of dissolved or dispersed 5-FU and/or leucovorin in accord with the doses disclosed herein. The dosage form also does not contain a preservative or bacteriostatic agent. An appropriate volume of the dosage form can be administered to achieve a desired therapeutic dose.

The dosage form also can comprise a first container comprising the picoplatin solution and a second container comprising a solution of leucovorin. The two containers can further comprise means to simultaneously administer the contents to a patient, e.g., the containers can be plastic intravenous bags that can be independently connected to a single intravenous tube so that the content of each container can be simultaneously administered to the patient, e.g., via a Y-link These containers can be packaged together with instructions regarding their end-use, e.g., in a kit.

In various embodiments, the invention provides a method for selecting a regimen of treatment for a patient afflicted with mCRC comprising: (a) identifying a patient afflicted with mCRC, (b) determining if the mCRC comprises a wild type K-ras gene or a mutated K-ras gene and (c) if the mCRC comprises a K-ras mutation positive genotype, then administering to the patient picoplatin and, optionally, 5-FU and leucovorin.

In one embodiment, if the patient fails first line treatment for colorectal cancer (e.g., wherein the cancer is refractory or wherein the cancer is progressive within six months of completing the regimen, wherein first line treatment can include platinum, such as oxaliplatinum), then the invention provides the use of about 5-150 mg/m² picoplatin (e.g., administered about every 7, 14, 21 or 28 days) in conjunction with a dose of about 5 to about 25 mg/kg, preferably about 10 mg/kg, bevacizumab (e.g., administered about every other week) and irinotecan (administered at e.g., about 50 to 150 mg/m², preferably about 125 mg/m² once weekly for 4 wk followed by 2 wk of rest; subsequent cycles can include administering irinotecan once weekly for 4 wk, followed by 2 wk of rest; based on response and adverse reactions, the dose may be adjusted in 25 to 50 mg/m² increments; the weekly dose may be increased to about 150 mg/m² or decreased to about 50 mg/m²) to treat colorectal cancer in a patient afflicted with colorectal cancer, including to treat metastatic colorectal cancer in a patient afflicted with colorectal cancer.

### DETAILED DESCRIPTION OF THE INVENTION

The use of picoplatin to treat metastatic colorectal cancer will be conducted in three parts. Phase 1 is a dose escalation study to identify the maximum tolerated dose (MTD) of picoplatin that can be administered either every two weeks or every four weeks, with 5-FU and leucovorin (LV) administered every two weeks, as initial therapy for subjects with metastatic colorectal cancer who have not been previously treated for metastatic disease. Phase 2 is a randomized study. In one arm of the study, picoplatin is tested at the MTD and selected schedule (every four weeks) combined with 5-FU and leucovorin that are administered every two weeks, to assess safety and efficacy. In the other arm, picoplatin will be substituted for oxaliplatin in a modified FOLFOX 6 regimen wherein the 100 mg/m² oxaliplatin dose in FOLFOX 6 has been reduced to 85 mg/m², and administered every 2 weeks, so that the two agents can be compared in the context of a widely used regimen. It is believed that cancer patients can be more effectively treated with the regimen of the present invention, which employ picoplatin instead of cisplatin, carboplatin or oxaliplatin, because they will experience fewer side effects, such as neuropathy, while preferably receiving higher doses of the platinum (Pt) drug.

Subjects eligible for the Phase 1 study will have Stage IV colorectal cancer and will have received no systemic therapy for metastatic cancer. Prior adjuvant chemotherapy with a 5-FU-based treatment regimen not containing oxaliplatin or irinotecan is acceptable if there has been a treatment-free interval of at least 6 months.

### Phase 1

Subjects are assigned centrally to treatment with picoplatin administered either every two or every four weeks and are assigned a dose of picoplatin to be given dependent on the study results to date. Each patient also receives 5-FU and leucovorin therapy every two weeks. Cohorts of 3 subjects receive their assigned dose of picoplatin and leucovorin and 5-FU according to the following schedule:

Day 1: Picoplatin, assigned dosage, as a 2-hour infusion, given either every cycle of 5-FU and leucovorin (q 2 weeks, Schedule A) or with every other cycle of 5-FU and leucovorin (q 4 weeks, Schedule B). Leucovorin, 400 mg/m² in D5W (water-5% dextrose), will be administered as a 2 hour infusion, either alone or, if the patient is to receive picoplatin, at the same time as picoplatin in separate bags using a Y-line. The leucovorin (±picoplatin) will be followed by a 5-FU bolus=400 mg/m² and then by 5-FU, 2,400 mg/m² in D5W administered as a 46 hour continuous infusion.

Subjects in Phase 1 are centrally assigned to one of two schedules of picoplatin. The first cohort of q 2 week (Schedule A) subjects are treated with picoplatin at a dosage of 45 mg/m², every cycle, q 2 weeks. Subsequent sequential cohorts of subjects assigned to this schedule receive picoplatin at dose levels increasing by 15 mg/m² if treatment is well tolerated to 75 mg/m² and then by 5 mg/m² to 90 mg/m², and finally by 15 mg/m² to 135 mg/m² until unacceptable dose-limiting toxicity (DLT) establishes the MTD.

The MTD is defined as the dose of picoplatin below the dose at which at least one third of at least 6 subjects experience a DLT. Tolerance data from only the first 4 weeks of treatment is used to determine the MTD. Thus, data following the first two doses of picoplatin in the q 2 week (Schedule A) subjects and following only the first dose of picoplatin in the q 4 week (Schedule B) subjects are considered. The first cohort of q 4 week (Schedule B) subjects will be treated with picoplatin at a dosage of 60 mg/m², every other cycle, q 4 weeks. Subsequent sequential cohorts of subjects assigned to this schedule will receive picoplatin at dose levels increasing by 30 mg/m² if treatment is well tolerated and until unacceptable dose-limiting toxicity (DLT) establishes the MTD. Depending on the pattern and severity of toxicity observed, additional intermediate dose levels of either schedule of picoplatin administration may be studied.

Within each schedule, the cohort size is 3 subjects, and is expanded to 6-10 subjects if a DLT is observed. Within each cohort of each schedule, one patient is treated initially; if no DLT is observed within the following 4 weeks (2 drug cycles), the remaining two subjects may be treated. If a DLT is observed in the first patient within a cohort, whether or not to proceed with enrollment of additional subjects in the cohort will be determined on a case-by-case basis. All subjects within a q 2 week (Schedule A) cohort will have completed 2 cycles (a cycle=the 2-day treatment regimen and an additional 12-day follow-up period) prior to escalating the dose in the next cohort of subjects. All subjects within a q 4 week (Schedule B) cohort will have completed 1 cycle of the 2-day treatment regimen (which should include 5FU/leucovorin) and an additional 26-day follow-up period prior to escalating the dose in the next cohort of Schedule B subjects.

If no DLT is observed among the 3 subjects within a cohort, picoplatin dose escalation may proceed in the next cohort of that schedule of picoplatin. If one DLT is observed, the cohort size at the specified dose and schedule of picoplatin is expanded to 6-10 subjects. Additional subjects may be entered at any dosage level and schedule below the dose at which 2 of 6 have DLT to obtain additional safety or efficacy data.

### Phase 2

The dose of the Phase 2 component of this study is selected based on the dose intensity of picoplatin achieved on each dose and schedule, the number of cycles tolerated and a subjective assessment of the tolerability and safety profile of each dose and schedule and a preliminary assessment of response rate in accord with Phase 1. The schedule for Phase 2 is selected as Schedule B, the q 4 week schedule. The subjects (approximately 100 with metastatic CRC, at about 25 clinical sites) are randomized to the modified FOLFOX 6⁶ or to FOLPI-150.

The FOLPI regimen is as follows:

Picoplatin 150 mg/m², is administered with every alternate cycle of 5-FU and leucovorin (q 4 weeks, Schedule B) as a 2 hour infusion. Leucovorin (400 mg/m² in D5W) is administered every 2 weeks as a 2-hour infusion, either alone, or given at the same time as the picoplatin in a separate bag using a Y-line. The administration of leucovorin±picoplatin is followed by a 5-FU bolus of 400 mg/m² and then by 5-FU, 2400 mg/m² in D5W administered as a 46 hour continuous infusion.

The modified FOLFOX 6 regimen is as follows:

Oxaliplatin 85 mg/m², as a 2-hour infusion is administered every 2 weeks. Leucovorin (400 mg/m² in D5W) is administered every 2 weeks as a 2-hour infusion. Oxaliplatin is given at the same time as the leucovorin in a separate bag using a Y-line. The administration of leucovorin +oxaliplatin is followed by a 5-FU bolus of 400 mg/m² and then by 5-FU, 2400 mg/m² in D5W administered as a 46 hour continuous infusion.

Neuropathy assessment is performed at baseline and after every two cycles of therapy (approximately every month) by an independent neurologist. The subject and the neurologist are not informed whether the platinum infused is oxaliplatin or picoplatin. This assessment by the neurologist is used to determine the incidence of Grade 2 or greater peripheral neuropathy. In Phase 2, for the purpose of determining toxicity for dose reduction or study drug discontinuation, the treating physician performs a neurological assessment using the NCI CTCAE. These CTCAE criteria are used to determine the need to dose reduce prior to each cycle. The assessment of the neurologist is used for determination of the safety endpoint, the incidence of neuropathy, and is performed independently every other cycle using the protocol-specified neuropathy scale, but is not be used for dose modification. For all subjects, hematology and serum chemistry laboratory studies are obtained prior to each treatment cycle. Treatment cycles (5-FU and leucovorin±picoplatin or oxaliplatin depending on schedule) are repeated every 2 weeks, but may be delayed up to 2 weeks while awaiting recovery of clinical or laboratory abnormalities. Data from all cycles of treatment and cumulative toxicity are assessed for safety analysis.

Tumor evaluations will be done at baseline and after every 4th treatment of 5-FU/leucovorin (every 8 weeks, unless doses have been delayed) on study. The efficacy endpoint will include objective response rate according to RECIST criteria.²⁶ Duration of response, time to progression, progression-free survival, and overall survival are also evaluated. The demographics and study treatments are summarized in Table 1A, 1B and 1C, below:

TABLE 1A
Patient Demographics

| | | Q4W n = 18 | Q2W n = 55 |
|---|---|---|---|
| Age (in years) | Median (range) | 60 (34, 75) | 58 (42, 70) |
| Age Group | <65 | 67% | 75% |
| | ≧65 | 33% | 26% |
| ECOG PS | 0 | 39% | 24% |
| | 1 | 61% | 76% |
| Prior Chemotherapy | Yes | 6% | 16% |
| Original Site of Disease | Colon | 50% | 56% |
| | Rectum | 44% | 42% |
| | Colon + Rectum | 6% | 2% |
| # of Metastatic Sites | Median (range) | 2 (1-5) | 2 (1-5) |
| Sites of Metastatic | Liver | 72% | 86% |
| Disease | Peritoneum/pelvic/ | 33% | 24% |
| | abdomen | | |
| | Lung | 33% | 44% |
| | Lymph nodes | 44% | 40% |
| | Ascites | 22% | 13% |
| | Skin/soft tissue | 6% | 0 |
| | Pleural effusion | 17% | 4% |
| | Other | 0 | 6% |

### Selection of Picoplatin Dose

Picoplatin was generally tolerated in combination with other myelosuppressive chemotherapeutic agents in previous Phase 1 studies at doses of 120-150 mg/m² administered every 3 weeks, i.e., doses equivalent to 80-100 mg/m² every 2 weeks or 160-200 mg/m² administered every 4 weeks. None of these studies, however, studied picoplatin in combination with 5-FU and leucovorin. 5-FU/leucovorin is not generally myelotoxic and thus the doses of picoplatin selected as the initial starting doses in the dose escalation portions of the current study, i.e., 45 mg/m² every two weeks and 60 mg/m² every four weeks, were well below the expected MTDs of picoplatin administered on these schedules.

### Administration of Picoplatin

Investigational-site staff must use standard cytotoxic handling procedures when preparing picoplatin for administration. Picoplatin is supplied as a ready-to-use formulation. The contents of the vials must be transferred to a suitable bag for administration. The compatibility of the formulation with typical infusion equipment has been assessed, and results have established compatibility with EVA infusion bags, PVC infusion tubing, and polypropylene syringes when the materials are protected from light. PVC infusion bags are not recommended for administration of picoplatin.

The compatibility of the formulation with typical administration sets has been assessed, and limits of acceptability have been set as 8 hours in a covered infusion bag. The product is highly sensitive to light and should not be exposed to ambient light for more than 1 hour without light protection. The bag must be protected from light during preparation and administration at the time of use.

There is no preservative or bacteriostatic agent present in the picoplatin formulation. Therefore, picoplatin must be transferred under aseptic conditions. The solution must be completely used or discarded within 8 hours of introduction into an infusion bag. As with all platinum complexes, contact with aluminum should be avoided.

Picoplatin should be administered by peripheral vein or central line; it must not be given by the intramuscular or subcutaneous route. The starting dose will be calculated based on the body surface area from the height and weight of the patient. If the patient's weight changes by more than 10%, the treating physician must recalculate the body surface area and amend the dose.

Picoplatin should be administered over 2 hours. It should be administered concurrently with leucovorin, in separate bags using a Y-line, when the two drugs are to be given on the same day. These two drugs have been tested and shown to be compatible when administered in this manner.

Subjects also received anti-emetic therapy consisting of a 5-HT₃ receptor antagonist plus dexamethasone 30 minutes prior to a dose of picoplatin. Subjects may also receive anti-emetic therapy for several days following treatment, which may include oral lorazepam, prochlorperazine, or equivalent for up to 7 days, as clinically indicated for breakthrough nausea and/or vomiting.

### Guidance for Administration

Detailed guidance for administration of 5-FU and leucovorin are provided in the product labels. Briefly, leucovorin 400 mg/m² IV infusion in D5W will be administered over 2 hours at the same time as picoplatin (if picoplatin is to be given on that day), in separate bags using a Y-line, followed by a bolus of 5-FU=400 mg/m² and then by 5-FU 2,400 mg/m² in D5W (recommended) administered as a 46-hour continuous IV infusion.

### Dose Modifications

### Dose Modification of Picoplatin

Dose-reduction is mandatory if any of the following hematological events are observed during the previous cycle: absolute neutrophil count (ANC) <0.5×10⁹/L for at least 5 days; absolute neutrophil count <1.0×10⁹/L complicated with Grade ≧2 fever (>38.5° C.); platelet count <25×10⁹/L; not reaching a platelet count ≧100 X 10⁹/L and ANC ≧1.5×10⁹/L by Day 15.

Dose reduction is also required for any treatment events involving any treatment-related Grade 3 toxicity, any Grade 4 toxicity, or any renal toxicity or neurotoxicities as described below.

For subjects receiving picoplatin every 2 weeks, the dose reduction should be 15 mg/m²; for subjects receiving picoplatin every 4 weeks the dose reduction should be 30 mg/m².

### Dose Reduction in the Event of Serum Creatinine Changes

Serum creatinine should be measured before every dose of picoplatin. For subjects with abnormal serum creatinine, the dose of picoplatin (but not 5-FU or leucovorin) should be modified according to the following Table 2 in Phase 1:

TABLE 2

| | Dose modification for | Dose modification for |
|---|---|---|
| Serum | q 2 week (Schedule A) | q 4 week (Schedule B) |
| Creatinine Value | picoplatin subjects | picoplatin subjects |
| ≦institutional ULN | recommended dose | recommended dose |
| >1.0 to 1.5 times | reduce by 25% | reduce by 25% |
| ULN | | |
| >1.5 to 2.0 times | reduce by 50% | reduce by 50% |
| ULN | | |
| >2.0 times ULN | discontinue treatment | discontinue treatment |
| | with picoplatin | with picoplatin |

In Phase 2, the following dose reductions are recommended for elevated serum creatinine (Table 3):

TABLE 3

| | Dose modification for |
|---|---|
| Serum creatinine | Phase 2 FOLPI subjects |
| ≦institutional ULN | recommended dose |
| >1.0 to 1.5 times ULN | reduce by picoplatin 30 mg/m² |
| >1.5 to 2.0 times ULN | reduce by picoplatin 60 mg/m² |
| >2.0 times ULN | discontinue treatment with |
| | picoplatin |

### Dose Modification in the Event of Neurotoxicity

The dose of picoplatin should be modified according to the CTCAE grade of toxicity and its duration as shown on Table 4:

TABLE 4

| | Duration of Toxicity | |
|---|---|---|
| | Resolves | Persistent |
| Toxicity | before | (present at start of |
| Grade | next cycle | next cycle) |
| Grade 1 | No change | Maintain picoplatin dose |
| Grade 2 | No change | Reduce picoplatin dose by |
| | | 30 mg/m² |
| Grade 3 | Reduce | Discontinue picoplatin |
| | picoplatin dose | |
| | by 30 mg/m² | |

| | |
|---|---|
| Grade 4 | Discontinue picoplatin |

Up to three dose reductions of a 30 mg/m² may occur should toxicity not improve or worsen at a later cycle.

### Dose Modification of 5-FU

The first time the dose of picoplatin is reduced, the bolus dose of 5-FU should be omitted. The second time the dose of picoplatin is reduced, the infusional dose should be reduced by 600 mg/m². Once decreased, the reduced dose of 5-FU should be continued; i.e., the dose of 5-FU should not be subsequently increased.

If the platelet count or ANC count is Grade 1 or 2 at day 15 in a cycle with picoplatin, and the subject receives the alternate i.e., even numbered cycle that does not include picoplatin, the dose of 5-FU should not be reduced at this cycle. At the next treatment cycle, the doses of picoplatin and 5-FU should be reduced by one level. Dose modifications for Grade 3 or 4 non-hematological events must be made. Continue treatment only once toxicity has resolved to <Grade 3.

### Dose Modification of Leucovorin

There are no dose modifications for leucovorin, unless drug sensitivity is suspected because of a temporal relationship to the time of leucovorin administration.

### Results

The total picoplatin exposed in Phase 1 study is summarized below on Table 5.

TABLE 5

| | | | | | |
|---|---|---|---|---|---|
| | | | Cumulative | Dose | |
| | | | Dose, | Intensity, | |
| | | Cycles* | mg/m² | mg/m²/wk | Relative |
| | | (median, | (median, | (median, | Mean Dose |
| Dose Level | n | range) | range) | range) | Intensity (%) |

Q4W

| | | | | | |
|---|---|---|---|---|---|
| 60 mg/m² | 3 | 16 (4-20) | 301 (120-543) | 13 (8-15) | 87 |
| 90 mg/m² | 3 | 24 (14-28) | 755 (480-902) | 16 (13-18) | 83 |
| 120 mg/m² | 3 | 4 (4-24) | 240 (240-630) | 28 (12-29) | 86 |
| 150 mg/m² | 3 | 7 (4-11) | 670 (300-870) | 32 (31-32) | 81 |
| 180 mg/m² | 6 | 6 (2-46) | 386 (180-1470) | 35 (5-43) | 76 |

Q2W

| | | | | | |
|---|---|---|---|---|---|
| 45 mg/m² | 12 | 12 (2-30) | 343 (90-1144) | 16 (7-22) | 82 |
| 60 mg/m² | 3 | 5 (4-24) | 211 (210-966) | 20 (19-23) | 76 |
| 75 mg/m² | 3 | 10 (5-12) | 529 (331-826) | 24 (24-32) | 79 |
| 80 mg/m² | 1 | 30 | 1145 | 19 | 77 |
| 85 mg/m² | 5 | 8 (1-16) | 420 (85-659) | 23 (13-41) | 80 |
| 90 mg/m² | 10 | 8 (3-26) | 435 (210-960) | 23 (12-44) | 69 |
| 105 mg/m² | 12 | 8 (4-28) | 475 (270-1871) | 27 (12-52) | 67 |
| 120 mg/m² | 3 | 8 (3-12) | 668 (315-780) | 41 (26-44) | 73 |
| 135 mg/m² | 6 | 8 (4-15) | 480 (360-914) | 38 (11-58) | 70 |

*A cycle equals a dose of 5-FU/LV and a follow-up period of 2 weeks, with or without picoplatin.

73 patients have been treated to date in Phase 1. In the q 2 w schedule, 1 of 6 patients showed a DLT of Grade 4 thrombocytopenia and neutropenia at a picoplatin dose level of 105 mg/m². The MTD for the q 2 w schedule was determined to be 85 mg/m². In the q 4 w schedule, DLT was observed at 180 mg/m² in 2 of 6 patients. The MTD was determined to be 150 mg/m². Patients have received up to 24 cycles and the therapy was well tolerated.

For both schedules, dose delays were primarily from neutropenia or thrombocytopenia, with increased hematological toxicity observed at higher doses as shown on Table 6, below.

TABLE 6
Hematological Toxicity

| Lab | Worst Grade | Q4W (n = 18) | Q2W (n = 55) |
|---|---|---|---|
| ANC* | 1 | 0 | 2% |
| | 2 | 17% | 15% |
| | 3 | 22% | 38% |
| | 4 | 39% | 31% |
| Platelets | 1 | 22% | 35% |
| | 2 | 28% | 27% |
| | 3 | 17% | 15% |
| | 4 | 22% | 13% |
| Hemoglobin | 1 | 17% | 29% |
| | 2 | 44% | 46% |
| | 3 | 22% | 11% |
| | 4 | 6% | 6% |

*1 patient on each arm had grade 3 febrile neutropenia

Grade 3 non-hematological toxicities related to treatment are summarized on Table 7.

TABLE 7
Nonhematologic Adverse Events*

| | Q4W (n = 18) | | Q2W (n = 55) | |
|---|---|---|---|---|
| | All | Grade ¾ | All | Grade ¾ |
| Nausea | 50% | 0 | 56% | 2% |
| Vomiting | 22% | 0 | 18% | 0 |
| Anorexia | 22% | 0 | 27% | 0 |
| Neuropathy** | 22% | 0 | 22% | 0 |
| Asthenia | 17% | 0 | 36% | 2% |
| Alopecia | 17% | NA | 31% | NA |
| Fatigue | 17% | 0 | 7% | 0 |
| Creatinine Increased*** | 11% | 0 | 20% | 0 |
| Diarrhea | 17% | 0 | 9% | 0 |
| Dizziness | 0 | 0 | 16% | 0 |
| Stomatitis | 0 | 0 | 9% | 4% |
| Erythema | 6% | 0 | 4% | 0 |
| Tinnitus | 0 | 0 | 6% | 0 |

*Drug-related events that occurred in more than 1 patient.
**Neuropathy includes neuropathy, peripheral sensory neuropathy, neuropathy peripheral, and polyneuropathy
***When assessed and graded using lab values, 28% on the Q4W arm and 29% on the Q2W arm had grade 1 creatinine levels. 1 patient each on the Q2W arm had grade 1 and grade 2. There were no grade 3 or 4 creatinine levels.

The cardiac and stomatitis events were attributed to the 5-FU component. No Grade 2 or higher neuropathy has been reported, even for four patients who have received a cumulative picoplatin dose of greater than about 900 mg/m², e.g., from about 902-1871 mg/m², a surprising and unexpected result, particularly in view of a high incidence of moderate to severe neuropathy observed at comparable doses of oxaliplatin. Picoplatin can be safely administered with FU and LV without the dose limiting neuropathy associated with FOLFOX.

In Schedule A (picoplatin q 2 week), the preferred dosage range is about 45-120 mg/m², e.g., doses of 45 to105 mg/m², e.g., 85 mg/m².

In Schedule B (picoplatin q 4 week), the preferred dose can be higher, e.g., about 120-210 mg/m², e.g., 120-180 mg/m², e.g., 150 mg/m². A lower dose can also be administered, e.g., at 45-90 mg/m², e.g., 60 mg/m².

Of 73 evaluated subjects evaluated by CT scan there have been 19 confirmed partial responses and one complete response (unconfirmed). Fifty-five of the subjects of the Q2 week schedule have been evaluated and 13 partial responses were observed (24%). Surprisingly, some patients in cohort A1 (45 mg/m²) showed a partial response. Eighteen of 18 subjects in the Q4 week schedule have been evaluated and 6 partial responses were observed (33%). These results are summarized on Table 8.

TABLE 8
Tumor Assessment (RECIST)

| | Q4W (n = 18) | Q2W (n = 55) |
|---|---|---|
| Partial Response | 6 (33%) | 13 (24%) |
| Stable Disease | 6 (33%) | 28 (51%) |
| Disease Control (PR + SD) | 12 (67%) | 41 (75%) |
| Progressive Disease | 6 (33%) | 10 (18%) |
| Not evaluable | 0 | 4 (7%) |

One patient was a 47 year-old woman with CRC diagnosed 12 Nov. 07. She was treated with laparotomy and sigmoidostomy; four hepatic metastases were present, the largest was (31 mm). After 42 weeks of FOLPI, repeat spiral CT showed complete response, confirmed. At that time the investigator removed the patient from study for resection of her primary lesion.

Phase II Study Randomized Phase II study of picoplatin in combination with 5-fluorouracil and leucovorin (FOLPI) as a neuropathy-sparing alternative to mFOLFOX-6 as first line therapy for colorectal cancer. A randomized, controlled study comparing the safety and efficacy of Q4W FOLPI (FOLPI with a 4 week dosing interval for picoplatin) at the picoplatin maximum tolerated dose (MTD) of 150 mg/m² in comparison with an mFOLFOX-6 regimen of 85 mg/m² oxaliplatin Q2W in combination with 5-FU and leucovorin in patients with metastatic CRC and no prior chemotherapy was carried out. Efficacy was assessed by objective tumor response using RECIST), progression-free survival, and overall survival. The safety of each regimen was evaluated based on the incidence of adverse effects. Peripheral neuropathy was assessed by 3 methods: investigator (using CTCAE), blinded independent neurologist and patient assessment (FACT; neurotoxicity questionnaire). The independent neurologist blinded to the treatment assignment assessed peripheral neuropathy using the following scale: 0: none, i.e., no neurosensory symptoms; 1: paresthesias, dysesthesias that interfere with function but not activities of daily living and persist between cycles; 3: paresthesias, dysesthesias with pain or functional impairment that also interfere with activities of daily living and persist between cycles; and 4: paresthesias, dysesthesias that are persistent and are disabling or life threatening.

Study Demographics, are shown below on Table 9. Each arm of the study was comparable in size, and of comparable median age.

TABLE 9
Demographics

| | | FOLPI | mFOLFOX-6 |
|---|---|---|---|
| | | N = 51 | N = 50 |
| Age (in years) | Median (range) | 59 (25-78) | 62 (34-81) |
| Age Group | <65 years | 35 (71%) | 27 (54%) |
| | ≧65 years | 15 (29%) | 23 (46%) |
| ECOG PS | 0 | 5 (10%) | 10 (20%) |
| | 1 | 46 (90%) | 40 (80%) |
| Adjuvant Therapy | Yes | 4 (8%) | 5 (10%) |
| Original Site of Disease | Colon | 21 (41%) | 36 (72%) |
| | Rectum | 27 (53%) | 11 (22%) |
| | Colon + Rectum | 3 (6%) | 3 (6%) |
| Number of Metastatic | Median (range) | 2 (1-6) | 2 (1-6) |
| Sites | | | |
| Sites of Metastatic Disease | Liver | 43 (86%) | 43 (84%) |
| | Lymph nodes | 22 (43%) | 19 (38%) |
| | Lung | 20 (39%) | 15 (30%) |
| | Peritoneum/pelvic/abdominal | 11 (22%) | 10 (20%) |
| | Bone | 4 (8%) | 1 (2%) |
| | Ascites | 4 (8%) | 1 (2%) |
| | Skin/soft tissue | 1 (2%) | 1 (2%) |
| | Pleural Effusion | 0 | 2 (4%) |
| | Other | 9 (18%) | 9 (18%) |

### Results

101 pts were randomized (51 to FOLPI, 50 to FOLFOX-6). Pts have received a mean of 10 (median of 8), max 28 2-week cycles of FOLPI and a mean of 11 (median of 10), max 31 2-week cycles of FOLFOX-6. Median dose intensity of picoplatin (FOLPI)=28 mg/m²/wk (range 9-70; mean mg/m²/week of 29 (s.d. 6)); median relative dose intensity=83%. Median dose intensity of oxaliplatin (FOLFOX-6)=36 mg/m²/wk (range 17-65; mean mg/m²/week of 36 (s.d. 5)); median relative dose intensity=92%.

TABLE 10
Response

| | FOLPI | mFOLFOX-6 |
|---|---|---|
| | N = 51 | N = 50 |
| Complete Response | 2 (4%) | 3 (6%) |
| Partial Response | 9 (18%) | 11 (22%) |
| Stable Disease | 27 (53%) | 24 (48%) |
| Disease Control (PR + CR + SD) | 38 (75%) | 38 (76%) |
| Progressive Disease | 8 (16%) | 9 (18%) |
| Not evaluable | 5 (10%) | 3 (6%) |

Median progression free response (PFS) for FOLPI is 6.8 months (95% CI: 5.2, 7.4) and 7.0 months for FOLFOX (95% CI: 4.4, 8.5).

One patient was a 70 year-old woman with diagnosed CRC. She was treated with a/p resection and presented with a 20-mm hepatic metastasis. After 8 weeks of FOLPI treatment, repeat spiral CT was interpreted by the PI as complete response. Independent radiologist evaluation was partial response, confirmed.

Another patient was a 66 year old Caucasian female with history of ischemic cardiac disease, hypertension and stroke, who underwent sigmoidectomy for colorectal cancer. Two hepatic metastases measuring a total of 47 mm were present when she started treatment with FOLPI. To date, she has completed>25 two-week cycles of treatment over 1 year, achieving complete response by cycle 17 (approximately 34 weeks) after starting treatment. Adverse events included grade 3 thrombocytopenia and neutropenia, and alopecia. She continued on treatment in complete response.

Neurotoxicity (peripheral neuropathy) was observed in 60% of pts on FOLFOX (16% G 3/4) and 29% of pts on FOLPI (no G 3/4). Most frequent G 3/4 AEs on FOLPI were neutropenia (60%), thrombocytopenia (40%) and anemia (14%). In the FOLFOX arm, other than neuropathy, the most frequent G 3/4 AEs were neutropenia (20%) and thrombocytopenia (12%). Disease control (CR+PR+SD) was 75% for FOLPI and 76% for FOLFOX. In the FOLPI arm there were 2 CR (4%) and 9 PR (18%). In the

FOLFOX arm there were 3 CR (6%) and 11 PR (22%).

TABLE 10
Hematological Toxicity*

| | Grade | FOLPI N = 50 | mFOLFOX-6 N = 49 |
|---|---|---|---|
| ANC | 1 | 2 (4%) | 8 (16%) |
| | 2 | 8 (16%) | 6 (12%) |
| | 3 | 15 (30%) | 9 (18%) |
| | 4 | 17 (34%) | 3 (6%) |
| Platelets | 1 | 13 (26%) | 23 (47%) |
| | 2 | 10 (20%) | 2 (4%) |
| | 3 | 18 (36%) | 5 (10%) |
| | 4 | 5 (10%) | 4 (8%) |
| Hemoglobin | 1 | 14 (28%) | 24 (49)% |
| | 2 | 19 (38%) | 14 (29%) |
| | 3 | 10 (20%) | 3 (6%) |
| | 4 | 4 (8%) | 3 (6%) |

*1 patient had febrile neutropenia

TABLE 11
Nonhematologic Adverse Events

| | FOLPI | mFOLFOX-6 |
|---|---|---|
| | N = 51 | N = 50 |
| Nausea | 43% | 54% |
| Asthenia | 37% | 36% |
| Vomiting | 28% | 28% |
| Neuropathy | 29% | 60% |
| Anorexia | 18% | 12% |
| Diarrhea | 22% | 18% |
| Alopecia | 20% | 4% |
| Increase creatinine | 12% | 10% |

FOLPI and mFOLFOX-6 deliver almost identical dose intensities of platinum. Three measurements of neuropathy agree on the superiority of FOLPI as a neuropathy-sparing alternative to FOLFOX. The Q4W FOLPI regimen exhibits later and less frequent and severe neurotoxicity than does mFOLFOX-6, while producing comparable rates of progression free survival and disease control, such as partial response, complete response and stable disease. Neurotoxicity is not dose-limiting for the FOLPI regimen, and the Q4W FOLPI regimen has comparable non-neurological tolerability to mFOLFOX-6. Although thrombocytopenia and neutropenia (hematological toxicity) are more frequent and severe with FOLPI than with mFOLFOX-6, they are manageable, and the acute gastrointestinal toxicity of the two regimens, as well as ototoxicity, is similar. Thus, FOLPI is a neuropathy sparing alternative to mFOLFOX-6 as first-line therapy for colorectal cancer.

### Bibliography

1. Jemal et al., Cancer Statistics, 2004. CA Cancer J Clin 54(1): 8-29, 2004.
2. Hoff et al., Oncology (Huntingt) 18(6): 705-708, 2004.
3. Meyerhardt et al., N Engl J Med 352(5): 476-87, 2005.
4. Penland et al., Oncology (Huntingt) 18(6): 715-722, 2004.
5. Saltz et al., N Engl J Med, 343(13): 905-14, 2000.
6. Tournigand et al., J Clin Oncol, 22(2): 229-37, 2004.
7. de Gramont et al., J Clin Oncol, 18(16): 2938-47, 2000.
8. Rothenberg et al., J Clin Oncol, 21(11): 2059-69, 2003.
9. Andre et al., N Engl J Med, 350(23): 2343-51, 2004.
10. Hwang et al., In: Clinical Use of Oxaliplatin: Case Studies and Roundtable Discussion, Editor Marshall J, CMP Healthcare Media, Oncology Publishing Group, Manhasset, N.Y. 2004.
11. Douillard, J Y, Schiller, J., Eur J Cancer 38(Suppl 8): S25-S31, 2002.
12. Beale, P, et al., Br J Cancer 88(7): 1128-1134, 2003.
13. Raynaud F I, et al., Clin Cancer Res 3(11): 2063-2074, 1997.
14. Holford J, et al., Anticancer Drug Des 13(1): 1-18, 1998.
15. Holford J, et al., Br J Cancer 77(3): 366-373, 1998.
16. Rogers P, et al., Eur J Cancer 38(12):1653-1660, 2002.
17. Sharp S Y, et al., Eur J Cancer 38(17):2309-15, 2002.
18. Plasencia C, et al., Invest New Drugs 22(4):399-409, 2004.
19. Murakami H, et al., Eur J Cancer 38(Suppl 8): S1-S5, 2002
20. Giaccone G, et al., Eur J Cancer 38 (Suppl 8): S19-S24, 2002.
21. Gore M E, et al., Eur J Cancer 38(18): 2416-2420, 2002.
22. Treat J, et al., Eur J Cancer 38(Suppl 8): S13-18, 2002.
23. Perez R P, et al., Eur J Cancer 34(10): 1535-42, 1998.
24. Gelmon K A, et al., Ann Oncol 15(7):1115-22, 2004.
25. Gelmon K A, et al., National Cancer Institute of Canada-Clinical Trials Group trial, IND 129. Ann Oncol 14: 543-548, 2003.
26. Therasse P, et al., New Guidelines to Evaluate the Response to Treatment in Solid Tumors. European Organization for Research and Treatment of Cancer, National Cancer Institute of the United States, National Cancer Institute of Canada. J Natl Cancer Inst 92(3): 205-216, 2000.

U.S. Ser. No. 61/055,071, filed May 21, 2008;

U.S. Ser. No. 61/027,387, filed Feb. 8, 2008:

PCT/US2009/000770, filed Feb. 6, 2009;

U.S. Ser. No. 61/027,382, filed Feb. 8, 2008;

PCT/US2009/000773, filed Feb. 6, 2009;

U.S. Ser. No. 61/027,360, filed Feb. 8, 2008;

PCT/US2009/000750, filed Feb. 6, 2009;

U.S. Ser. No. 11/982,841, filed Nov. 5, 2007;

U.S. Ser. No. 61/170,487, filed Apr. 17, 2009;

PCT/US2008/008076; and

PCT/US2008/008669.

All publications, patents and patent applications cited herein are incorporated by reference herein.

1-26. (canceled)

27. A method of treatment of colorectal cancer, comprising:
administering to a patient afflicted with colorectal cancer effective amounts of a combination of picoplatin, bevacizumab, and irinotecan.

28. The method of claim 27, wherein the patient having colorectal cancer has previously been treated with a platinum regimen, wherein the cancer is refractory or wherein the cancer is progressive within six months of completing the regimen.

29. The method of claim 27, wherein the cancer is metastatic.
